(19) United States
(12) Patent Application Publication
(10) Pub. No.: US 2022/0404141 A1
(43) Pub. Date: Dec. 22, 2022

(54) 3D BODY SCANNER FOR CREATING 3D BODY MODELS

(71) Applicant: NAKED LABS AUSTRIA GMBH, Wien (AT)

(72) Inventors: Peter Kreuzgruber, Wien (AT); Gerhard Schultes, Wien (AT)

(21) Appl. No.: 17/845,216

(22) Filed: Jun. 21, 2022

(30) Foreign Application Priority Data
Jun. 21, 2021 (DE) 10 2021 115 999.7

(51) Int. Cl. G01B 11/22; G01G 19/50

(52) U.S. Cl. CPC G01B 11/22; G01G 19/50; H02J 50/10

(57) ABSTRACT

A 3D body scanner for creating 3D body models of an outer body shape of a person includes a scanner unit, which includes at least one depth sensor configured for spatially detecting a visual field. The scanner unit is powered by electrical energy, and a platform is powered by electrical energy and configured for accommodating the person. The 3D body scanner includes an energy transmission arrangement that is configured to contactlessly transmit electrical energy between the scanner unit and the platform.

### FIELD OF THE INVENTION

The present invention relates to a 3D body scanner for creating 3D body models, in particular an outer body shape, of a person, having a scanner unit, which includes at least one depth sensor for spatially detecting a visual field, and having a platform for accommodating the person.

### BACKGROUND OF THE INVENTION

For example, DE 20 2009 017 401 U1, which corresponds to U.S. Pat. No. 9,289,158, which is hereby incorporated herein in its entirety by this reference for all purposes, describes a detection device for detecting spatial shapes of bodies. The disadvantage thereof is that the detection device is inflexible with respect to use.

### OBJECTS AND SUMMARY OF THE INVENTION

The object of the present invention is to improve the related art.

The problem is solved by a 3D body scanner having the features of the independent patent claim described below along with the drawings.

The invention relates to a 3D body scanner for creating 3D body models of a person. The 3D body model can be an outer body shape, i.e., for example, a silhouette, of the person. For the sake of simplicity, the descriptor “3D” can be omitted in the following. It is clear, however, that the body scanner and the body model are the 3D body scanner and the 3D body model, respectively.

The body scanner includes a scanner unit, which includes at least one depth sensor for spatially detecting a visual field. The body model is created or ascertained with the aid of the scanner unit and with the aid of the depth sensor.

Moreover, the body scanner includes a platform for accommodating the person. The platform is utilized, for example, in particular for locating the person at a defined place.

According to the invention, the body scanner includes an energy transmission arrangement, by means of which the electrical energy can be contactlessly transmitted between the scanner unit and the platform. As a result, the platform can be used in a flexible manner. Wiring for transmitting energy between the scanner unit and the platform can be dispensed with as a result. In addition, the platform can therefore be flexibly positioned.

It is advantageous when the platform includes an electrical energy store. The electrical energy store is preferably chargeable by means of the energy transmission arrangement. The electrical energy store can include, for example, an accumulator. When the body scanner, for example, is not used, the energy store can be charged by means of the energy transmission arrangement. If the body scanner is then used, the platform is supplied with electrical energy from the energy store. Additionally or alternatively, energy can also be transmitted to the platform by means of the energy transmission arrangement.

It is advantageous when the energy transmission arrangement includes a transmitting unit for transmitting the electrical energy and a receiving unit for receiving the electrical energy. The scanner unit can include the transmitting unit and the platform can include the receiving unit. As a result, energy is transmitted from the scanner unit to the platform. Consequently, only the scanner unit can be connected to a grid. The platform is then disconnected from the grid. This also has an advantage in that the platform cannot become damaged in the event of an overvoltage in the grid.

It is advantageous when the scanner unit includes a base element, which preferably includes the transmitting unit. As a result, the transmitting unit is close to the ground, and so the energy transmission to the platform is effective.

It is advantageous when the scanner unit, in particular the base element, has a recess and the platform has an insertion section corresponding to the recess such that the platform can be arranged in the recess by means of the insertion section. As a result, errors in the use of the body scanner can be avoided. Once the insertion section has been inserted into the recess, the energy transmission takes place, for example, to charge the energy store. An erroneous orientation of the platform with respect to the scanner unit, in which energy transmission cannot take place, is therefore avoided.

It is advantageous when the scanner unit, in particular the base element, includes the transmitting unit in the area of the recess and the platform includes the receiving unit in the insertion section. Alternatively, the scanner unit, in particular the base element, can include the receiving unit in the area of the recess and the platform can include the transmitting unit in the insertion section.

It is advantageous when the scanner unit includes a coupling arrangement such that the platform can be coupled to the scanner unit for the purpose of energy transmission. Additionally or alternatively, the platform can include the coupling arrangement such that the platform can be coupled to the scanner unit for the purpose of energy transmission. The certainty that the energy transmission takes place is increased as a result thereof as well. The platform and the scanner unit remain connected to each other by means of the coupling arrangement and cannot become detached from each other.

It is advantageous when the transmitting unit and the receiving unit can be arranged one above the other congruently in a horizontal direction of the scanner unit for the purpose of energy transmission. The energy transmission therefore takes place in the vertical direction and, in fact, in an effective way. As a result, the energy transmission arrangement can be designed to be space-saving.

It is advantageous when the transmitting unit is arranged under the receiving unit when the 3D body scanner is positioned as intended. As a result, good effectiveness with respect to the energy transmission can be achieved.

It is advantageous when the energy transmission arrangement is designed such that the electrical energy can be inductively transmitted. Higher powers can be transmitted with the aid of inductive energy transmission. Additionally or alternatively, it is advantageous when the energy transmission arrangement is designed such that the electrical energy can be capacitively transmitted. Capacitive energy transmission is simpler to design with respect to the construction.

It is advantageous when the transmitting unit includes a transmitting coil and the receiving unit includes a receiving coil, wherein the transmitting coil and the receiving coil are arranged coaxially to each other for the purpose of energy transmission. As a result, inductive energy transmission can be formed. Additionally or alternatively, the transmitting unit and the receiving unit can also include an appropriate capacitor arrangement for forming the capacitive energy transmission.

It is advantageous when the 3D body scanner includes at least one 3D sensor. The depth sensor can be designed, for example, as the 3D sensor. Additionally or alternatively, the depth sensor and/or the 3D sensor can also include at least two cameras in order to be able to detect the person by means of triangulation. Additionally or alternatively, the depth sensor can also be based on structured light, on LIDAR, and/or radar.

It is advantageous when the platform includes a weighing unit. In addition to detecting the body shape of the person, a weight of the person can therefore also be ascertained. Additionally or alternatively, the platform can also include a body fat sensor, however, by means of which a body fat percentage of the person can be measured, for example, with the aid of a resistance measurement of the body of the person. Additionally or alternatively, moreover, the platform can also include a pulse sensor or other sensors for ascertaining a physical condition, however. Additionally or alternatively, the platform can also be designed as a rotary table. In order to be able to capture a complete 3D image of the person, i.e., the complete 3D body shape, the person can be turned with the aid of the platform as a rotary table in order to be able to turn all sides of the person toward the scanner unit. The person can therefore continue to stand still. The platform or the rotary table can include an electric motor in order to be able to turn the person. The weighing unit and/or the rotary table are/is preferably supplied with electrical energy by means of the energy transmission unit. In the process, the energy store of the platform can also first be charged with the aid of the energy transmission unit.

It is also advantageous when the scanner unit and the platform each include a radio interface, so that a wireless radio link can be formed between the scanner unit and the platform. As a result, information and/or commands can be transmitted between the platform and the scanner unit. For example, an instruction to turn the person can therefore be transmitted when the platform is designed as a rotary table. Additionally or alternatively, however, weight information can also be transmitted from the weighing unit or the body fat sensor to the scanner unit.

It is advantageous when the scanner unit includes a mirror. As a result, the person can observe him/herself.

It is advantageous, moreover, when the body scanner includes a control unit, which carries out the energy transmission, the detection of the person, the data transmission between the platform and the scanner unit, and/or the evaluation of the gathered data. In particular, the control unit can also ascertain the body shape of the person on the basis of the data gathered by means of the depth sensor.

### BRIEF DESCRIPTION OF THE DRAWINGS OF EXEMPLARY EMBODIMENTS

Further advantages of the invention are described in the following exemplary embodiments, wherein:

FIG. 1 shows a schematic representation of a body scanner with the person, and

FIG. 2 shows a schematic sectional view of the platform coupled to the scanner unit, including an energy transmission arrangement.

### DETAILED DESCRIPTION OF EXEMPLARY EMBODIMENTS OF THE INVENTION

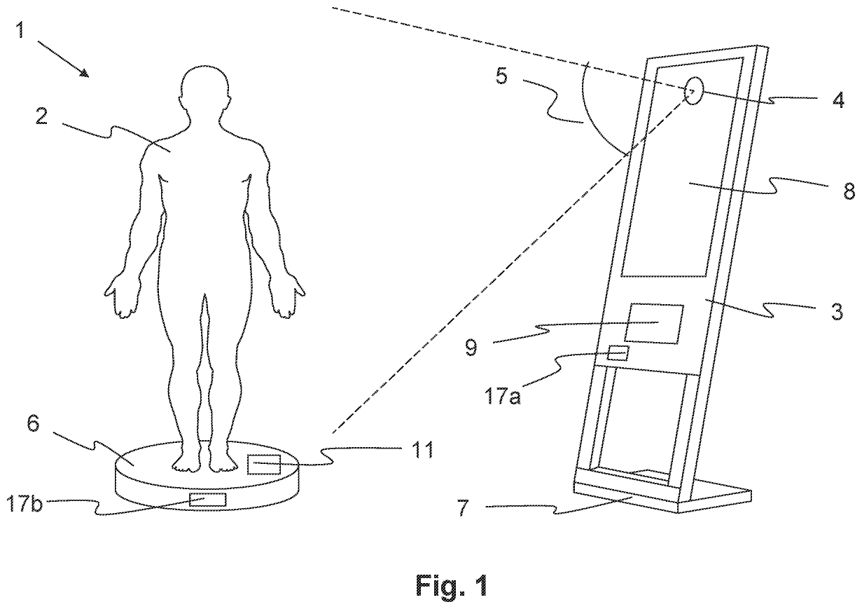

FIG. 1 shows a schematic representation of a body scanner 1 with the person 2. With the aid of the body scanner 1, 3D body models, in particular an outer shape or a silhouette, of the person 2 can be created. The body model can be utilized, for example, in the clothing industry in order to be able to fit or determine the suitability of garments. In addition, the body models can also be utilized for medical purposes, for documenting athletic progress, for creating computer models of the person, etc.

Moreover, the body scanner 1 includes a scanner unit 3, which is powered by electrical energy and includes at least one depth sensor 4 for detecting the person. The person 2 can be detected in a visual field 5 with the aid of the depth sensor 4. The depth sensor 4 gathers depth data of the visual field 5, on the basis of which the body model is ascertained. The at least one depth sensor 4 gathers, for all intents and purposes, 3D data of the visual field 5.

Moreover, the body scanner 1 includes a platform 6 for accommodating the person 2. The person 2 can stand on the platform 6, wherein a location of the person 2 is predefined by means of a placement of the platform 6.

The platform 6 is powered by electrical energy and can be designed, for example, as a rotary table, and so the person 2 can be turned in order to be able to scan the person 2 from all sides. The platform 6 can include an electric motor for this purpose.

Additionally or alternatively, the platform 6 can also include a weighing unit, by means of which a weight of the person 2 can be recorded. Additionally or alternatively, the platform 6 can include a sensor for detecting body parameters. In addition to the aforementioned weighing unit, the platform 6 can also include, additionally or alternatively, a body fat sensor, a pulse sensor, etc.

Moreover, as shown here, the scanner unit 3 can include a base element 7 in order to set the scanner unit 3 on an underlying surface.

Moreover, the scanner unit 3 of the present exemplary embodiment includes a mirror 8, with the aid of which the person 2 can observe him/herself.

In addition, the body scanner 1 according to the present exemplary embodiment includes a control unit 9. In this exemplary embodiment, the scanner unit 3 includes the control unit 9. The body scanner 1 can be controlled with the aid of the control unit 9. For example, the control unit 9 is configured to evaluate the data gathered by means of the depth sensor 4 and create the body model. Additionally or alternatively, the control unit 9 can be configured to control the platform 6.

The 3D body scanner 1 also includes an energy connection (not shown here) in order to supply the 3D body scanner 1 with electrical energy, for example, from a grid. For example, the scanner unit 3 includes the energy connection.

According to the present exemplary embodiment, the platform 6 includes an energy store 11 in order to be able to supply the platform 6 with energy. The energy store 11 can be rechargeable. For example, the energy store 11 includes an accumulator.

According to the present exemplary embodiment, the scanner unit 3 and the platform 6 each include a radio interface 17a, 17b, respectively, and so the two radio interfaces 17a, 17b can communicate wirelessly and/or exchange data with each other. The two radio interfaces 17a, 17b can be designed, for example, as Bluetooth.

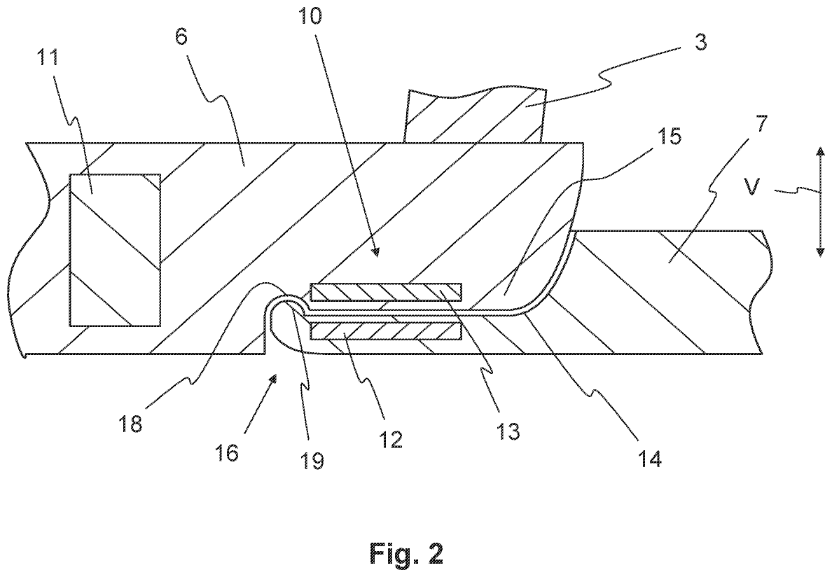

FIG. 2 shows a schematic sectional view of the platform 6 coupled to the scanner unit 3, including an energy transmission arrangement 10.

Electrical energy can be contactlessly exchanged between the scanner unit 3 and the platform 6 by means of the energy transmission arrangement 10. As schematically shown in FIG. 2 for example, during the contactless exchange of electrical energy, the scanner unit 3 and the platform 6 are not in physical contact with each other. As a result, the body scanner 1 can be designed in a simple way. In addition, the platform 6 for accommodating the person 2 can be more flexibly positioned.

Moreover, the energy store 11 can be charged with the aid of the energy transmission arrangement 10 in order to be able to autonomously operate the platform 6. The energy store 11 can include, for example, an accumulator.

According to the present exemplary embodiment, the energy transmission arrangement 10 transmits the electrical energy when the platform 6 is coupled to the scanner unit 3. Moreover, the body scanner 1 includes a coupling arrangement 16 in order to couple the platform 6 to the scanner unit 3. As a result, it can be ensured that the platform 6 remains coupled to the scanner unit 3 during the transmission of energy. The coupling arrangement 16 includes, according to the present exemplary embodiment, a coupling recess 18, into which a coupling element 19 engages. The coupling arrangement 16 can couple the platform 6, for example, in a form-locking manner, with the scanner unit 3. Additionally or alternatively, the coupling arrangement 16 can also couple the platform 6 to the scanner unit 3 in a force-locked manner.

Moreover, the energy transmission arrangement 10 according to the present exemplary embodiment includes a transmitting unit 12 and a receiving unit 13. Electrical energy can be transmitted with the aid of the transmitting unit 12 and the electrical energy can be received with the receiving unit 13.

According to the present exemplary embodiment, the scanner unit 3 includes the transmitting unit 12 and the platform 6 includes the receiving unit 13. Additionally or alternatively, the scanner unit 3 can also include a receiving unit 13 and the platform 6 can include a transmitting unit 12.

The transmitting unit 12 and the receiving unit 13 can be designed, for example, as a transmitting coil and a receiving coil, respectively, and so the electrical energy can be inductively transmitted. Additionally or alternatively, the energy transmission arrangement 10 can also be designed such that the electrical energy is capacitively transmitted. In that case, the transmitting unit 12 and the receiving unit 13 can be appropriate capacitor elements.

Moreover, the scanner unit 3, in particular the base element 7, has a recess 14, into which the platform 6 can be inserted with an insertion section 15. As a result, it can be ensured that the energy transmission arrangement 10 can transmit the electrical energy. In particular, it is ensured that the transmitting unit 12 and the receiving unit 13 are arranged correctly with respect to each other.

In addition, the platform 6 is guided toward the scanner unit 3 from the direction in which the mirror 8 shown in FIG. 1 is arranged. The platform 6 is therefore guided toward and coupled to the scanner unit 3 from the direction in which the depth sensor 4 is arranged. The platform 6 is therefore guided toward and coupled to the scanner unit 3 from the front.

According to the present exemplary embodiment schematically shown in FIG. 2, the transmitting unit 12 and the receiving unit 13 are arranged one above the other. That is, the transmitting unit 12 and the receiving unit 13 are arranged spaced apart from each other in a vertical direction V.

The present invention is not limited to the represented and described exemplary embodiments. Modifications within the scope of the claims are also possible, as is any combination of the features, even if they are represented and described in different exemplary embodiments.

### LIST OF REFERENCE CHARACTERS

1 3D body scanner
2 person
3 scanner unit
4 depth sensor
5 visual field
6 platform
7 base element
8 mirror
9 control unit
10 energy transmission arrangement
11 energy store
12 transmitting unit
13 receiving unit
14 recess
15 insertion section
16 coupling arrangement
17 radio interface
18 coupling recess
19 coupling element
V vertical direction

What is claimed is:

1. A 3D body scanner for creating 3D body models of an outer body shape of a person, the 3D body scanner comprising:
a scanner unit, which is powered by electrical energy and includes a depth sensor configured for spatially detecting a visual field;
a platform powered by electrical energy, configured for accommodating the person and spaced apart from the scanner unit;
an energy transmission arrangement, by means of which the electrical energy can be contactlessly transmitted between the scanner unit and the platform.

2. The 3D body scanner of claim 1, wherein the platform includes an electrical energy store.

3. The 3D body scanner of claim 1, wherein the energy transmission arrangement includes a transmitting unit configured for transmitting the electrical energy and a receiving unit configured for receiving the electrical energy, wherein the scanner unit includes the transmitting unit and the platform includes the receiving unit.

4. The 3D body scanner of claim 3, wherein the scanner unit includes a base element, which includes the transmitting unit.

5. The 3D body scanner of claim 4, wherein the base element includes a recess with a defined configuration, and wherein the platform includes an insertion section corresponding to the defined configuration of the recess such that the platform can be arranged in the recess via receipt of the insertion section into the recess.

6. The 3D body scanner of claim 4, wherein the transmitting unit is disposed in the immediate vicinity of the recess and the platform includes the receiving unit in the insertion section.

7. The 3D body scanner of claim 1, wherein the scanner unit and/or the platform include(s) a coupling arrangement that is configured such that the platform can be coupled to the scanner unit in a manner that permits energy transmission from the scanner unit to the platform.

8. The 3D body scanner of claim 3, wherein the transmitting unit and the receiving unit can be arranged one above the other congruently in a horizontal direction of the scanner unit in a manner that permits energy transmission from the scanner unit to the platform.

9. The 3D body scanner of claim 3, wherein the transmitting unit is arranged under the receiving unit.

10. The 3D body scanner of claim 1, wherein the energy transmission arrangement is configured for transmitting the electrical energy inductively and/or capacitively.

11. The 3D body scanner of claim 3, wherein the transmitting unit includes a transmitting coil and the receiving unit includes a receiving coil, wherein the transmitting coil and the receiving coil are arranged coaxially to each other for the purpose of energy transmission.

12. The 3D body scanner of claim 1, wherein the scanner unit includes a 3D sensor.

13. The 3D body scanner of claim 1, wherein the platform includes a weighing unit.

14. The 3D body scanner of claim 1, wherein each of the scanner unit and the platform includes a respective radio interface that is configured to permit a wireless radio link to be formed between the scanner unit and the platform.

15. The 3D body scanner of claim 1, wherein the scanner unit includes a mirror.

16. The 3D body scanner of claim 4, wherein the receiving unit is disposed in the immediate vicinity of the recess in the base element of the scanner unit, and the platform includes the transmitting unit in the insertion section.

17. The 3D body scanner of claim 1, wherein the platform includes a rotary table.
